(19) United States
(12) Patent Application Publication
(10) Pub. No.: US 2003/0176801 A1
(43) Pub. Date: Sep. 18, 2003

(54) Audio/ECG sensor/coupler with integrated signal processing

(75) Inventors: Peter M. Galen, Portland, OR (US); David B. Swedlow, Danville, CA (US); Damon J. Coffman, Portland, OR (US); Robert A. Warner, Tigard, OR (US)

Correspondence Address: Robert D. Varitz, ROBERT D. VARITZ, P.C., 2007 S.E. Grant Street, Portland, OR, 97214, US

(73) Assignee: Inovise Medical, Inc.

(21) Appl. No.: 10/389,530

(22) Filed: Mar. 14, 2003

Related U.S. Application Data
(60) Provisional application No. 60/364,770, filed on Mar. 14, 2002.
(60) Provisional application No. 60/364,405, filed on Mar. 14, 2002.

(51) Int. Cl. A61B 5/0402

(52) U.S. Cl. 600/513; 600/528

(57) ABSTRACT

Small-scale sensor structure attachable to a person's anatomy for collecting and conveying physiologically-related electrical and audio signals, including the conveying of processed output signals that relate input electrical and audio signals.

### CROSS REFERENCE TO RELATED APPLICATIONS

This application claim priority to two co-pending U.S. Provisional Patent Applications. One of these applications, filed Mar. 14, 2002 bears Serial No. 60/364,770, and is entitled “ECG/Sound Algorithm Adapter”. The other application, filed Mar. 14, 2002, bears Serial No. 60/364,405 and is entitled “Combined ECG/Sound Assessment-Call Algorithm”.

### INTRODUCTION

This invention pertains to apparatus and methodology for the collection and conveyance to external monitoring structure of a combination of anatomically-acquired, physiologically-related electrical and audio signals, such as heart-related signals. In particular, it relates to such apparatus and methodology which utilize a small sensor that is attachable directly to a person's anatomy, with that sensor possessing an internal data processor that implements and invokes an algorithm which is effective to produce a sensor output signal that integrates this dual-nature information for monitoring and assessment purposes.

While it will be apparent, and should be understood, that the apparatus and methodology of this invention can be useful with regard to various kinds of physiological signals, it has special utility in dealing with heart-related ECG and audio signals. Accordingly the invention description which now follows presents a focus on the monitoring of heart-related activity.

Among some of the significant features and offerings of this invention is the fact that it is designed structurally with a body-attachable sensor device which is capable of performing important data processing that relates audio and ECG electrical signals which are gathered “out at the location”, so-to-speak, of a small sensor unit which is attached to the anatomy during use. This arrangement enables the sensor structure of this invention to feed and present processed audio and ECG electrical information outwardly to otherwise substantially conventional heart-monitoring circuit devices which, because the invention produces data processing at the location of the device which is attached to the anatomy, does not typically require any hardware modification in order to be compatible with the sensor structure of the invention. It thus becomes easily retro-useable with much conventional external monitoring gear which may, at most, only need relatively modest internal software changes to make full use of the information provided by the structure and methodology of this invention. It thus offers an arrangement where much of the important signal processing that needs to be done, in order effectively to utilize the kind of information gathered by this sensor, is done out at the location of the sensor per se. Outward conveyance or transmission of information from the sensor structure may take place either by wire, or wirelessly.

These and other important features and advantages that are offered by the present invention will become more fully apparent as the description which now follows is read in conjunction with the accompanying drawings.

### DESCRIPTION OF THE DRAWINGS

FIG. 1 is simplified block/schematic diagram illustrating a preferred and best mode embodiment of present invention.

FIG. 2 is a fragmentary view illustrating a modified form of the arrangement shown in FIG. 1.

FIG. 3 is a simplified block/schematic/circuit diagram which is readable from two different points of view, and specifically from respective points of view which relate it, on the one hand, to the invention embodiment pictured in FIG. 1, and on the other hand, to the modified form of the invention shown in FIG. 2. Toward the right side of FIG. 3, there appears a vertical dash-dot line which is related to this dual point of view capability of FIG. 3. Very specifically, all structure that is shown in FIG. 3 relates directly to the modification of the invention shown in FIG. 1. With respect to the modification illustrated in FIG. 2, those components illustrated in FIG. 3 which are to the right of the just-mentioned vertical dash-dot line are not present in the version illustrated in FIG. 2.

FIG. 4 is a simplified graphical illustration of a discernable output provided by the sensor configuration pictured in FIGS. 1 and 3.

FIG. 5 generally illustrates an implementation of the invention which conveys information “outwardly” from the sensor structure to external monitoring apparatus wirelessly.

### DETAILED DESCRIPTION OF THE INVENTION

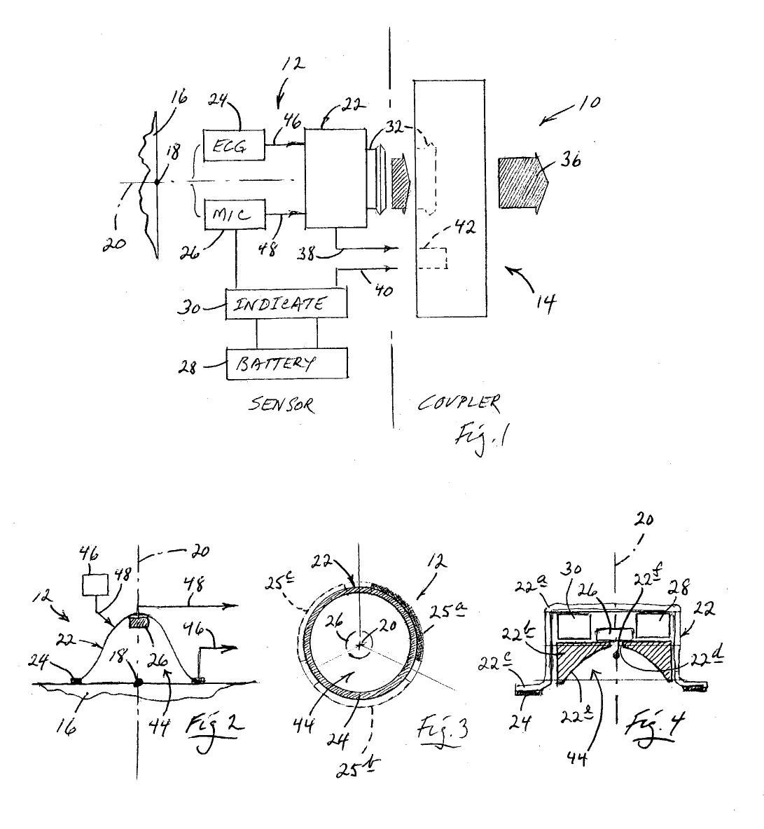

Turning now to the drawings, and referring first of all to FIGS. 1 and 3, indicated generally at 10 is sensor structure constructed in accordance with the present invention. This structure, while, as mentioned above, being employable in relation to a variety of physiologically-related electrical and audio signals, is specifically discussed hereinbelow in conjunction with collecting from a person's anatomy, and conveying subsequently, related ECG-electrical-signal, and heart-related audio-signal, information. Pictured in these two drawing figures is the preferred and best mode embodiment of this sensor structure.

Included in sensor structure 10 are sensor hardware 12, signal-collection structure 14, computer-based algorithmic structure 16, and a single output terminal 18. All of this structure preferably is formed as part of a relatively small unit which is adapted to be suitably attached to a selected site on a person's anatomy for the purpose of collecting both ECG and audio signals, which result from the heart activity of a person. Hardware 12 is intended effectively to be tethered at the outboard end of suitable electrical conductor structure which may extend as a cable toward, and connect with, various different kinds of conventional, external, heart-activity monitoring structure (not shown).

Included in signal-collection structure 14 are two electrically conductive ECG electrodes, also referred to herein collectively as a pair of ECG electrodes, 20, 22, and an audio transducer in the form of a small microphone 24. While different specific arrangements of these signal collection devices may be employed, preferably, electrode 20 takes the form of an annular ring which has a central axis that is shown in dash-dot markings at 26 in FIG. 1, with microphone 24 being spaced to the right of this electrode as seen in FIG. 1, and positioned on and along axis 26. These two coaxial devices are preferably further arranged in such a manner that microphone 24 sits within a shrouding volume possessing a curved, parabolic surface that is effectively aimed, so-to-speak, to the left in FIG. 1 along axis 26. The precise arrangement of electrode 20 with respect to microphone 24 is not critical at all to the present invention, but has been found to provide a collection geometry which is quite effective. A co-pending regular U.S. patent application fully describing this arrangement bears Ser. No. ______ and was filed on ______, for “Method and Apparatus for Detecting and Transmitting Electrical and Related Audio Signals from a Single, Common Anatomical Site”. For the purposes of disclosure herein, the entire contents of that co-pending patent application are hereby incorporated fully by reference. The specification and drawings in this prior application are attached hereto as Appendix A.

Electrode 20 and microphone 24 can be thought of as being aimed along axis 26 toward a selected site 28 on a person's anatomy, shown generally and fragmentarily at 30. Site 28 herein is preferably that site on the anatomy with respect to which a conventional, so-called V-4 lead sits for collecting ECG information.

Electrode 22 may be of any suitable conventional construction, and this electrode, in the invention embodiment now being described, is positioned relatively closely adjacent electrode 20, whereby the central distance between these two electrodes, shown at D in FIG. 1 might typically about 1-inches. Focusing attention especially on FIG. 3, electrode 20, the V-4 electrode, feeds an ECG electrical output signal directly to a buffer and gain amplifier 32, and also via a conductor 34 to a signal combiner shown at 36.

Electrode 22 feeds its output signal, which is also an electrical ECG signal, to another buffer and gain amplifier 38. Electrode 22, with the device of this invention in use, contacts the anatomy site at a location which is referred to herein as V_{special}.

Buffer and gain amplifiers 32, 38 are conventional in construction, and have their outputs connected to the two inputs of a conventional difference amplifier 40, whose output terminal is coupled to one input in a conventional analog-to-digital converter 42. Amplifier 40 is also referred to herein as difference detecting circuitry. The output signal provided by amplifier 40 contains useful information relevant to the timing difference which may exist between related portions of the respective output signals furnished by electrodes 20, 22.

Microphone 24, which is suitably powered, or biased, by an onboard battery (not shown) feeds its output signal derived from heart-produced sounds to another input terminal in analog-to-digital converter 42. The output of converter 42 is connected to what is referred to herein as computer-based algorithmic structure 16 (mentioned earlier), inside of which is an appropriate data processor 44 which invokes and implements an algorithm 46 whose function herein will be more fully described shortly. The left side of algorithmic structure 16 in FIG. 3 is referred to as the input side, and the right side is referred to as the output side. An output signal generated by structure 16 is referred to herein as an algorithmically processed output signal.

Difference amplifier 40 is also referred to herein as difference-detecting circuitry.

Completing a description of what is shown in FIG. 3, the output side of structure 16 is connected as shown to a conventional digital-to-analog converter 48, whose output is connected to previously mentioned signal combiner 36. The output of signal combiner 36 is connected to previously mentioned output terminal 18.

For the purpose of discussing the embodiment of the sensor structure of this invention so far specifically discussed, the vertical dash-dot line toward the right side of FIG. 3, and the two right-pointing, large, darkened triangles, need not be read in order to understand how this version of the invention is constructed and is employed.

When the device of this invention is placed on a person's anatomy at the appropriate location with, for example, axis 26 intersecting a specific site on the anatomy, which site is that site normally chosen for attachment of a conventional ECG V-4 lead, electrode 22 also contacts the anatomy, and microphone 24 is positioned to “listen” for heart-produced audio sounds that flow to it along axis 26. There is a slight timing difference which exists between comparable locations on the detected electrical waveforms experienced by electrodes 20, 22, and this difference is detected by difference amplifier 40 which produces a timing-related output signal that is fed to analog-to-digital converter 42. Audio signals output from microphone 24 are also fed to the analog-to-digital converter which, in turn, feeds all such information as a digital data-stream to the input side of algorithmic structure 16. Conductor 34 feeds V-4 ECG electrical lead information directly to combiner 36 which, through digital-to-analog converter 48, receives the algorithmically processed output signal produced by structure 16 in response to the infeed of information from converter 42.

All of the information thus arriving as input information to signal combiner 36 is output in a manner which effectively produces a discernable signal that looks something like that which is pictured in FIG. 4 in the drawings. Here one can see, shown at 50, a waveform which looks very much like a traditional, conventional ECG electrical waveform. This waveform rises and falls above a zero axis shown at 52, and the signal progresses in the manner of elapsing time as indicated at T in FIG. 4.

Superimposed on waveform 50 in accordance with the implementation of algorithm 46 herein, and in response to data received from the two ECG electrodes and from the microphone, are spike signals which are shown at A, B, C and D in FIG. 4. As can be seen, spikes A, B and C are positive-going, and occur in time at locations which are indicated at tA, tB, tC, respectively. Spike D is a negative-going spike which occurs at a timing location shown at tD. With respect to two of these spikes, namely spikes C and D, their respective amplitudes are marked generally in FIG. 4 as AMPC and AMPD.

These spikes give, among other things, important timing information with respect to the occurrences of audio activity that has been detected by microphone 24, and the time locations, the total number, the polarities, the amplitudes, and the time densities of these spikes can yield important information about heart behavior as these spikes are read in conjunction with their locations, etc., on and with respect to waveform 50.

The invention embodiment thus so far described delivers what is referred to herein as a total sensor output signal over a single conductor (or terminal) 18.

Considering now the modified form of this invention mentioned earlier herein, such being illustrated both in FIG. 2, and in relation to a “visual modification” of FIG. 3, as indicated by the vertical dash-dot line pictured in that figure, this modification differs from the first described embodiment of the invention by the omission of signal combiner 36 and of digital-to-analog converter 48. In this embodiment of the invention, sensor structure 10 possesses two output terminals shown at 54, 56. Output terminal 56 here provides conventional ECG V-4 signal lead information which is fed outwardly to external monitoring apparatus in a very conventional manner. Terminal 56 is therefore what can be thought of as a single-signal, dedicated terminal whose full communication bandwidth is available to that signal. The output signal presented on output terminal 54, which is also feed outwardly to appropriate external monitoring structure, contains the digital version of the algorithmically produced output signal generated directly by structure 16. In other words, output terminal provides the digital version of the analog signal pictured in FIG. 4. This second embodiment of the invention thus delivers a toal sensor signal over two conductors (or terminals).

Both implementations of the sensor structure of this invention, and both optional methodologies respectively offered by these two implementations, offer important, through slightly different, and albeit clearly overlapping, advantages.

The FIG. 1/FIG. 3 modification offers sophisticated audio/ECG signal processing “out” at the site of the sensor, and “single-output-conductor-only” communication to external monitoring structure which will require no hardware modification (only software) to receive and interpret information arriving by this conductor. Electrical-potential reference for this output conductor may be conventionally furnished.

The FIG. 2/FIG. 3 modification, which also offers the same decided advantage of furnishing single-site, outboard signal-processing functionality, introduces a second output conductor, but does so in a fashion whereby the direct ECG output conductor (56 in FIG. 3) is essentially dedicated with full bandwidth to signals provided by electrode 20.

While the final, or overall, sensor output signal may be conveyed or transmitted outwardly to external monitoring apparatus by a wired connection, FIG. 5 illustrates a modest modification of the invention which employs, at the location of the sensor/coupler structure, an appropriate wireless transmitter 58, of any suitable category, which receives transmittable information or indicated generally by shaded arrow 60. This shaded arrow represents an in-feed of signal information from conductors and terminals such as those shown at 18, 54, 56 in FIGS. 1, 2 and 3.

Accordingly, while preferred and best mode features of the structure and methodology of the present invention have been described and illustrated herein, it is appreciated that variations and modifications may be made without departing form the sprit of the invention. One should also be reminded that, while details of the structure and operation of this invention have been described herein in the special context of monitoring heart-related activity, the invention's scope is to be understood to embrace monitoring generally of various, similar, physiologically-related electrical and audio signals.

We claim:

1. Sensor structure for collecting from a person's anatomy, and for conveying, related electrical-signal, and physiologically-related audio-signal, information comprising
sensor hardware attachable to a selected site in a person's anatomy,
signal-collection structure carried by said hardware and including at least a pair of body electrical-signal-collecting electrodes, and at least one audio transducer, each operable, during use of the sensor structure to collect associated, physiologically-related information from the anatomy, and to produce a related, respective output signal, and
computer-based algorithmic structure also carried by said hardware and having an input side operatively coupled to each of said electrodes and said at least one audio transducer to receive output signals therefrom, said algorithm structure also having an output side, and being operable, upon the receipt of such output signals, to create at its output side an algorithmically processed output signal which is effectively deliverable to external monitoring structure, and which contains a selected presentation of aspects of the respective output signals received from said electrodes and said at least one audio-transducer.

2. The sensor structure of claim 1 which further includes difference-detecting circuitry operatively interposed said electrode pair and the input side of said algorithmic structure, operable to furnish said algorithmic structure's input side with information relating to a timing difference extant between related portions of the respective output signals produced by said electrodes.

3. The sensor of claim 2, wherein said algorithmic structure is constructed to include, in its created algorithmically-processed output signal, information relating to such a timing difference.

4. The sensor structure of claim 2, wherein said algorithmic structure includes a data processor.

5. The sensor structure of claim 2 which further includes a signal combiner operatively connected both the said algorithmic structure's said output side, and to one of said electrodes, operable to produce a sensor output signal which is deliverable directly to external monitoring structure.

6. The sensor structure of claim 5, wherein said signal combiner is structured in such a manner that the mentioned sensor output signal combines information derived both from the algorithmically processed output signal and from the output signal produced by said one electrode.

7. The sensor structure of claim 5, wherein said sensor output signal is discernable in the form generally of an ECG waveform decorated with audio-event-related spikes having at least one of the characteristics including populations, time-densities, timing locations, amplitudes, and polarities.

8. The sensor structure of claim 1 which further includes an output terminal which is operatively connected to receive directly an output signal produced by one only of said electrical-signal-collecting electrodes.

9. A method utilizing a small-scale, body-attachable sensor structure for collecting and conveying related physiologically-related electrical and audio signals comprising
at the location of such an attached sensor structure, collecting, from a pair of spaced anatomical sites, a pair of such electrical signals, and from at least one of the sites in that pair of sites, a related audio signal,
within the sensor structure, applying signal-processing to a selection of such collected electrical and audio signals to create an algorithmically processed signal which contains presentable aspects of the selected electrical and audio signals, and then
effectively delivering to external monitoring structure, and at least as a part of total output information so delivered by the sensor structure, a sensor output signal which contains information based upon such an algorithmically processed output signal.

10. The method of claim 9, wherein delivering of such a total output signal takes the form of delivery over a single output conductor.

11. The method of claim 9, wherein delivering of such a total output signal takes the form of delivery over a pair of output conductors.

12. The method of claim 9, wherein delivering of such a total output signal takes the form of wireless communication.
